(19) United States
(12) Patent Application Publication
(10) Pub. No.: US 2011/0009429 A1
(43) Pub. Date: Jan. 13, 2011

(54) HETEROCYCLIC COMPOUNDS AS INHIBITORS OF CXCR2

(76) Inventors: Paul Oakley, Horsham (GB); Neil John Press, Horsham (GB); Carsten Spanka, Basel (CH); Simon James Watson, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/867,569

(22) PCT Filed: Feb. 25, 2009
(86) PCT No.: PCT/EP09/52211

§ 371 Date: Aug. 13, 2010

(30) Foreign Application Priority Data
Feb. 26, 2008 (EP) 08151952.2

(51) Int. Cl. A61K 31/519; C07D 487/04; A61P 37/08; A61P 29/00; A61P 11/00

(52) U.S. Cl. 514/259.31; 544/263; 544/281; 514/259.3

(57) ABSTRACT

The present invention relates to compounds of formula (I)

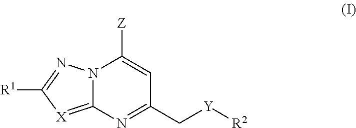

wherein R¹, R², X, Y and Z are as defined in the specification.

The present invention relates to organic compounds, e.g. compounds of formula (I), and uses thereof.

In one aspect the present invention provides a compound of formula

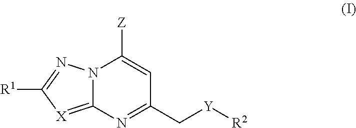

wherein
R¹ is a group of the formula: -A-(C₀-C₈ alkylene)-B;
A is a bond, —C(O)N(R^{a})—, —C(O)NHS(O)—, —C(O)NHS(O₂)—, —C(O)—, —C(O)O—, —C(O)-(5 or 6-membered N-bonded heterocyclic bridging group)-, —N(R^{a})C(O)—, —(CH₂)_{z}—N(R^{a})—, —(CH₂)_{z}—N(R^{a})S(O)—, —(CH₂)_{z}—N(R^{a})S(O₂)—, —C(═N—OR^{a})— or —NHC(═NH)N(R^{a})—;
B is H, OH, CN, NO₂, halogen, C₁-C₈ alkylthio, C₂-C₆ alkenyl, C₂-C₆ alkynyl, C₃-C₈ cycloalkyl, C₅-C₈ cycloalkenyl, C₆-C₁₄ aryl, a 5-10 membered heterocyclic group containing one or more heteroatoms selected from N, O and S, C₁-C₆ alkoxy, O—C₃-C₈ cycloalkyl, O—C₁-C₃ alkylene-C₃-C₈ cycloalkyl, O—C₆-C₁₄ aryl, O-benzyl, O-(5-10 membered heterocyclic group containing one or more heteroatoms selected from N, O and S), C(O)R^{d}, C(O)OR^{b}, OC(O)R^{b}, C(O)NR^{b}R^{b}, N(R^{b})C(O)R^{d}, NR^{b}R^{C}, S(O)C₁-C₆ alkyl or S(O₂)C₁-C₈ alkyl, wherein the alkyl, alkenyl and alkynyl groups are each optionally substituted by OH, halo or C₁-C₃ alkoxy, wherein the cycloalkyl and cycloalkenyl groups are each optionally fused to a benzene ring and the ring as a whole is optionally substituted by OH, halo, NH₂ or C₁-C₃ alkoxy, and wherein the aryl and heterocyclic groups are each optionally substituted by one or substituents each independently selected from OH, halo, NH₂, CN, NO₂, oxo, C₁-C₆ alkyl, C₁—O₆ hydroxyalkyl, C₁-C₆ haloalkyl, C₃-C₈ cycloalkyl, C₁-C₆ alkoxy, C₁-C₆ haloalkoxy, phenyl, a 5-10 membered heterocyclic group containing one or more heteroatoms selected from N, O and S and CO₂R^{b};
the (C₀-C₈ alkylene group) may be branched (e.g. —CH(CH₃)— or —CH(C₂H₈)—) and is optionally substituted by OH or C₁-C₃ alkoxy;
z is 0, 1, 2 or 3;
R^{a} and R^{b} are each independently selected from H, C₁-C₆ alkyl, C₃-C₈ cycloalkyl and C₅-C₈ cycloalkenyl;
R^{c} and R^{d} are each independently selected from H, C₁-C₆ alkyl, C₃-C₈ cycloalkyl, C₅-C₈ cycloalkenyl, C₆-C₁₄ aryl, a 5-10 membered heterocyclic group containing one or more heteroatoms selected from N, O and S, S(O)C₁-C₆ alkyl and S(O₂)C₁-C₆ alkyl, provided that R¹ is not hydrogen;
R² is C₆-C₁₄ aryl, —C₁-C₆ alkylene-C₆-C₁₄ aryl or a 5-10 membered heterocyclic group containing one or more heteroatoms selected from N, O and S, wherein the aryl and heterocyclic groups are each optionally fused to a 5 or 6-membered non-aromatic carbocyclic group or a 5 or 6-membered non-aromatic heterocyclic group containing one or more heteroatoms selected from N, O and S and wherein the ring systems are optionally substituted by OH, halo, NH₂, CN, NO₂, oxo, C₁-C₆ alkyl, C₁-C₆ hydroxyalkyl, C₁-C₆ haloalkyl, C₃-C₈ cycloalkyl, C₁-C₆ alkoxy, C₁-C₆ haloalkoxy, phenyl, a 5-10 membered heterocyclic group containing one or more heteroatoms selected from N, O and S and CO₂R^{b};

### X is C or N;

### Y is O or CH₂;

### Z is OR³ or NR³R⁴;

R³ is H, C₁-C₆ alkyl, C₃-C₈ cycloalkyl or C₅-C₈ cycloalkenyl;
R⁴ is H, C₁-C₆ alkyl, C₃-C₈ cycloalkyl and C₅-C₈cycloalkenyl, wherein the alkyl and cycloalkyl groups are each optionally substituted by one or more groups selected from OH and C₁-C₃ alkoxy;
or a pharmaceutically acceptable salt or solvate thereof.

An embodiment of the present invention provides a compound of formula (I) wherein

R¹, X, Y and Z are as defined anywhere herein and
R² is phenyl, optionally fused with a 5 membered non-aromatic heterocyclic group containing 2 oxygen heteroatoms, wherein the ring system is optionally substituted by one or more groups selected from C₁-C₃ alkyl and halogen; or
pyridinyl optionally substituted by one or more halogen atoms.

In another embodiment, the present invention provides a compound of formula (I) wherein

R¹, X, Y and Z are as defined anywhere herein and
R² is phenyl, optionally fused with a 5 membered non-aromatic heterocyclic group containing 2 oxygen heteroatoms, wherein the ring system is optionally substituted by one or more groups halogen.

In another embodiment, the present invention provides a compound of formula (I) wherein

R¹, R², Y and Z are as defined anywhere herein and

### X is C.

If not otherwise defined herein
alkyl includes linear or branched C₁-C₈ alkyl, such as C₁-C₆ alkyl or C₁-C₄ alkyl, e.g. methyl, ethyl, propyl, isopropyl, butyl, isobutyl, sec-butyl, etc., including unsubstituted or substituted alkyl, e.g. alkyl substituted by groups which are conventional in organic chemistry, e.g. halogen, OH, NH₂, C₁-C₆ alkoxy or halo-C₁-C₆ alkyl,
alkylene includes a hydrocarbon linking group containing the defined number of carbon atoms, which may be linear or branched and which may be unsubstituted or substituted, such as methylene (—CH₂—), 1,2-ethylene (—CH₂CH₂—), 1,1-ethylene (—CH(CH₃)—), 1,1-propylene (—CH(C₂H₅)—), 1,2-propylene (—CH₂CH(CH₃)—), 1,3-propylene (—CH₂CH₂CH₂—), etc.,
cycloalkyl includes C₃-C₈ cycloalkyl, such as C₃-C₆ cycloalkyl, e.g. cyclopropyl, cyclobutyl, cyclopentyl, cyclohexyl, etc.,
halogen includes fluoro, chloro, bromo, iodo, e.g. fluoro, chloro, bromo, preferably fluoro or chloro,
alkoxy includes C₁-C₆ alkoxy, such as C₁-C₄ alkoxy, e.g. methoxy, ethoxy, propyloxy, etc.,
alkylthio includes C₁-C₈ alkylthio, such as C₁-C₄ alkylthio, e.g. methylthio,
aryl includes C₆-C₁₄ aryl, e.g. phenyl, and fused systems where the aromatic carbocycle is fused to a heterocyclic group having 5 or 6 ring members and 1 to 4 heteroatoms selected from N, O, S, e.g. phenyl fused with 1,3-dioxolane,
heterocyclyl or heterocyclic includes heterocyclyl having 5 to 10 ring members and 1 to 4 heteroatoms selected from N, O, S, preferably N, O. The ring system may be alicyclic or aromatic, thus, heterocyclic includes heteroaryl. Example heterocyclic systems include heterocyclic groups having 5 or 6 ring members and 1 to 2 heteroatoms selected from N, O, S, e.g. pyridinyl, furanyl. The heterocyclic group may be fused to a carbocyclic ring, e.g. benzofused ring systems, or it may include a heterocyclic ring fused to a second heterocyclic ring, provided that the ring system as a whole satisfies the requirement for the defined number of ring atoms.
carbocycle includes a hydrocarbon ring system containing the defined number of carbon atoms. The ring may be saturated, partially unsaturated or aromatic and it may be substituted or unsubstituted.

In another embodiment, the present invention provides a compound of formula I selected of the group consisting of:
5-[2-(2-Fluoro-phenyl)-ethyl]-2-methyl-pyrazolo[1,5-a]pyrimidin-7-ol,
2-Amino-5-[2-(2-fluoro-phenyl)-ethyl]-[1,2,4]triazolo[1,5-a]pyrimidin-7-ol,
2-Benzyl-5-[2-(2-bromo-phenyl)-ethyl]-pyrazolo[1,5-a]pyrimidin-7-ol,
2-Benzyl-5-[2-(2-chloro-phenyl)-ethyl]-pyrazolo[1,5-a]pyrimidin-7-ol,
5-[2-(2-Bromo-phenyl)-ethyl]-2-furan-2-yl-pyrazolo[1,5-a]pyrimidin-7-ol,
5-[2-(2-Chloro-phenyl)-ethyl]-2-furan-2-yl-pyrazolo[1,5-a]pyrimidin-7-ol,
5-[2-(2,3-Difluoro-phenyl)-ethyl]-2-furan-2-yl-pyrazolo[1,5-a]pyrimidin-7-ol,
5-[2-(2,3-Difluoro-phenyl)-ethyl]-2-methyl-pyrazolo[1,5-a]pyrimidin-7-ol,
2-Benzyl-5-[2-(2,3-difluoro-phenyl)-ethyl]-pyrazolo[1,5-a]pyrimidin-7-ol,
5-(2-Chloro-phenoxymethyl)-2-methyl-pyrazolo[1,5-a]pyrimidin-7-ol,
5-(2-Bromo-phenoxymethyl)-2-methyl-pyrazolo[1,5-a]pyrimidin-7-ol,
5-(2,3-Difluoro-phenoxymethyl)-2-methyl-pyrazolo[1,5-a]pyrimidin-7-ol,
5-(2,4-Dichloro-phenoxymethyl)-2-methyl-pyrazolo[1,5-a]pyrimidin-7-ol,
5-(2,6-Difluoro-phenoxymethyl)-2-furan-2-yl-pyrazolo[1,5-a]pyrimidin-7-ol,
5-(6-Chloro-2-fluoro-3-methyl-phenoxymethyl)-2-furan-2-yl-pyrazolo[1,5-a]pyrimidin-7-ol
2-Furan-2-yl-5-(2-nitro-phenoxymethyl)-pyrazolo[1,5-a]pyrimidin-7-ol,
5-(2-Chloro-pyridin-3-yloxymethyl)-2-furan-2-yl-pyrazolo[1,5-a]pyrimidin-7-ol,
5-(2-Chloro-6-methyl-phenoxymethyl)-2-furan-2-yl-pyrazolo[1,5-a]pyrimidin-7-ol
2-Furan-2-yl-5-phenoxymethyl-pyrazolo[1,5-a]pyrimidin-7-ol.
5-(2,6-Dichloro-phenoxymethyl)-2-furan-2-yl-pyrazolo[1,5-a]pyrimidin-7-ol,
5-(Benzo[1,3]dioxol-4-yloxymethyl)-2-furan-2-yl-pyrazolo[1,5-a]pyrimidin-7-ol,
5-(2,3-Difluoro-phenoxymethyl)-2-furan-2-yl-pyrazolo[1,5-a]pyrimidin-7-ol,
5-(2-Chloro-phenoxymethyl)-2-furan-2-yl-pyrazolo[1,5-a]pyrimidin-7-ol,
2-Furan-2-yl-5-(2,4,6-trifluoro-phenoxymethyl)-pyrazolo[1,5-a]pyrimidin-7-ol,
2-Methyl-5-(2,3,5,6-tetrafluoro-phenoxymethyl)-pyrazolo[1,5-a]pyrimidin-7-ol,
2-Benzyl-5-(2,3,5,6-tetrafluoro-phenoxymethyl)-[1,2,4]triazolo[1,5-a]pyrimidin-7-ol.

Compounds of formula I in free or pharmaceutically acceptable salt form are hereinafter referred to alternatively as compounds of the invention.

According to formula I, the above-described embodiments and/or suitable features of the invention may be incorporated independently, collectively or in any combination. Thus, the term “an embodiment of the invention as defined anywhere herein” should be taken to mean “an embodiment of the invention as defined anywhere herein in any embodiment or aspect” and it should be clear to the skilled person that specific features of different embodiments or aspects can be combined within the scope of the invention as defined herein.

Compounds of formula I that contain a basic centre are capable of forming acid addition salts, particularly pharmaceutically acceptable acid addition salts. Pharmaceutically acceptable acid addition salts of the compound of formula I include those of inorganic acids, for example, hydrohalic acids such as hydrofluoric acid, hydrochloric acid, hydrobromic acid, hydroiodic acid, nitric acid, sulfuric acid, phosphoric acid; and organic acids, for example aliphatic monocarboxylic acids such as formic acid, acetic acid, trifluoroacetic acid, propionic acid and butyric acid, caprylic acid, dichloroacetic acid, hippuric acid, aliphatic hydroxy acids such as lactic acid, citric acid, tartaric acid or malic acid, gluconic acid, mandelic acid, dicarboxylic acids such as maleic acid or succinic acid, adipic acid, aspartic acid, fumaric acid, glutamic acid, malonic acid, sebacic acid, aromatic carboxylic acids such as benzoic acid, p-chloro-benzoic acid, nicotinic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid, ethanesulfonic acid, ethane-1,2-disulfonic acid, 2-hydroxy-ethanesulfonic acid, (+) camphor-10-sulfonic acid, naphthalene-2-sulfonic acid, naphthalene-1,5-disulfonic acid or p-toluenesulfonic acid. These salts may be prepared from compounds of formula I by known salt-forming procedures. Pharmaceutically acceptable solvates are generally hydrates.

Compounds of formula I which contain acidic, e.g. carboxyl groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine, arginine, benethamine, benzathine, diethanolamine, 4-(2-hydroxyethyl)morpholine, 1-(2-hydroxyethyl)pyrrolidine, N-methyl glucamine, piperazine, triethanolamine or tromethamine. These salts may be prepared from compounds of formula I by known salt-forming procedures. Compounds of formula I that contain acidic, e.g. carboxyl groups may also exist as zwitterions with the quaternary ammonium centre.

Compounds of formula I in free form may be converted into salt form, and vice versa, in a conventional manner. The compounds in free or salt form can be obtained in the form of hydrates or solvates containing a solvent used for crystallisation. Compounds of formula I can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Many compounds of the invention contain at least one asymmetric carbon atom and thus they exist in individual optically active isomeric forms or as mixtures thereof, e.g. as racemic mixtures. In cases where additional asymmetric centres exist the present invention also embraces both individual optically active isomers as well as mixtures, e.g. diastereomeric mixtures, thereof.

The invention includes all such forms, in particular the pure isomeric forms. The different isomeric forms may be separated or resolved one from the other by conventional methods, or any given isomer may be obtained by conventional synthetic methods or; by stereospecific or asymmetric syntheses. Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

The invention includes all pharmaceutically acceptable isotopically-labelled compounds of formula I wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen e.g. ²H and ³H, carbon e.g. ¹¹C, ¹³C and ¹⁴C, chlorine e.g. ³⁶Cl, fluorine e.g. ¹⁸F, iodine e.g. ¹²³I and ¹²⁵I, nitrogen e.g. ¹³N and ¹⁵N, oxygen e.g. ¹⁸O, ¹⁷O and ¹⁸O, and sulfur e.g. ³⁵S.

Certain isotopically-labelled compounds of formula I, for example those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with heavier isotopes such as deuterium (²H) may afford certain therapeutic advantages that result from greater metabolic stability, for example increased in vivo half-life or reduced dosage requirements, and hence may be preferred in some circumstances. Substitution with positron emitting isotopes, such as ¹¹C, ¹⁸F, ¹⁵O, and ¹³N can be useful in Positron Emission Topography (PET) studies for examining substrate receptor occupancy.

Isotopically-labelled compounds of formula I can generally be prepared by conventional techniques known to those skilled in the art or by processes analogous to those described in the accompanying examples using an appropriate isotopically-labelled reagent in place of the non-labelled reagent previously used.

Pharmaceutically acceptable solvates in accordance with the invention include those wherein the solvent of crystallisation may be isotopically substituted e.g. D₂O, d₆-acetone or d₆-DMSO.

The present invention also includes tautomers of a compound of the present invention, e.g. a compound of the present invention where Z is OH may be present in the following forms:

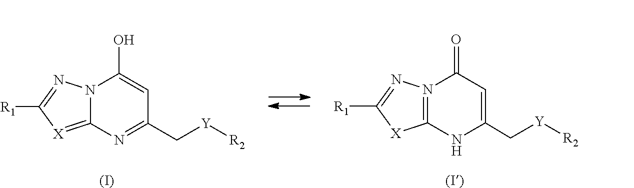

Any compound described herein, e.g. a compound of the present invention, may be prepared as appropriate, e.g. according, e.g. analogously, to a method as conventional, e.g. or as specified herein. Starting materials are known or may be prepared according, e.g. analogously, to a method as conventional or as described herein.

A compound of formula I thus obtained may be converted into another compound of formula I, e.g. or a compound of formula I obtained in free form may be converted into a salt of a compound of formula I and vice versa.

Any compound described herein, e.g. a compound of the present invention, may be prepared as appropriate, e.g. according, e.g. analogously, to a method as conventional, e.g. or as specified herein. Starting materials are known or may be prepared according, e.g. analogously, to a method as conventional or as described herein.

In another aspect the present invention provides a process for the preparation of a compound of the present invention comprising reacting a compound of formula

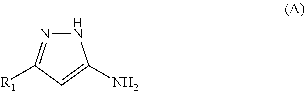

with a compound of formula

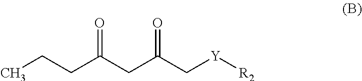

under appropriate conditions, e.g. in the presence of acetic acid at 110° C. for 1 to 2 hours, to obtain a compound of formula (I) of the invention; OR
reacting a compound of formula

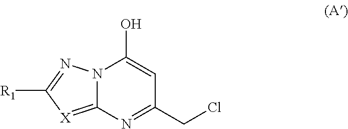

with a compound of formula R₂OH under appropriate conditions, e.g. in the presence of DMF and NaH, to obtain a compound of formula (I) of the invention.

A compound of formula I thus obtained may be converted into another compound of formula I, e.g. or a compound of formula I obtained in free form may be converted into a salt of a compound of formula I and vice versa.

Compounds of the invention, are useful as pharmaceuticals.

Accordingly the invention also provides a compound of formula I in free or pharmaceutically acceptable salt form for use as a pharmaceutical.

In another aspect the present invention provides the use of a compound of formula (I) wherein the substituents are as defined above as a pharmaceutical.

The compounds of the invention act as CXCR2 receptor antagonists, thereby inhibiting the infiltration and activation of inflammatory cells, in particular neutrophils, monocytes and CD8+ T cells and mediators involved in chronic obstructive pulmonary disease (COPD). The compounds of the invention therefore provide symptomatic relief and reduce disease progression.

The airways of subject with COPD exhibit an inflammatory response which is predominantly neutrophilic. When the airways are exposed to cigarette smoke macrophages, CD8+ T cells and epithelial cells are activated and release pro-inflammatory mediators, oxidants, cytokines and neutophilic chemotactic factors, IL-8, GROα, ENA-78 and leukotrienes. IL-8, GROα and ENA-78 are selective chemoattractants for neutrophils. In human neutrophils IL-8 binds two distinct receptors with similar affinity, CXCR1 and CXCR2. Closely related chemokines including GROα, β, γ, NAP-2 and ENA-78 bind only to CXCR2. Inhibiting neutrophil recruitment is therefore a recognised therapeutic strategy for treating several lung diseases. Blocking the binding of IL-8, GROα and ENA-78 to the chemokine receptor CXCR2 can provide beneficial effects in patients with COPD by suppressing the infiltration and activation of key inflammatory cells, thereby reducing subsequent tissue damage, mucus secretion, airflow obstruction and disease progression.

The IL-8 and GROα chemokine inhibitory properties of compounds of the invention can be demonstrated in the following ASSAYS:

Receptor Binding Assay [¹²⁵I] IL-8 (human recombinant) are obtained from Amersham Pharmacia Biotech, with specific activity 2000 Ci/mmol. All other chemicals are of analytical grade. Human recombinant CXCR2 receptor expressed in Chinese hamster ovary cells (CHO-K1) is purchased from Euroscreen. The Chinese hamster ovary membranes are prepared according to protocol supplied by Euroscreen. Membrane protein concentration is determined using a Bio-Rad protein assay. Assays are performed in a 96-well micro plate format according the method described in White, et al., J Biol. Chem., 1998, 273, 10095). Each reaction mixture contains 0.05 mg/ml CXCR2 membrane protein in 20 mM Bis-Tris-propane, pH 8.0, containing 1.2 mM MgSO₄, 0.1 mM EDTA, 25 mM NaCl and 0.03% CHAPS. In addition, compound of interest pre-dissolved in dimethylsulphoxide (DMSO) so as to reach a final concentration of between 10 μM and 0.0005 μM (final concentration of DMSO 2% (v/v)) is added. Binding is initiated by addition of 0.02 nM ¹²⁵I-IL-8. After 2 hours at room temperature the plate is harvested using a Brandell™ 96-well harvester onto glass fibre filter plate (GF/c) blocked with 1% polyethyleneimine+0.5% BSA and washed 3 times with 25 mM NaCl, 10 mM TrisHCl, 1 mM MgSO₄, 0.5 mM EDTA, 0.03% CHAPS, pH 7.4. The filter is dried at 50° C. overnight. Backseal is applied to the plate and 50 μl of liquid scintillation fluid added. The counts are measured on the Packard Topcount™ scintillation counter.

### [³⁵S]-GTPγS Binding Assay for Human CXCR2 Receptor Using SPA Technology

[³⁵S]-GTPγS (with specific activity 1082 Ci/mmol) and wheat germ agglutinin poly vinyl toluene scintillation proximity beads are purchased from Amersham Pharmacia Biotech. The Chinese hamster ovary cell (CHO-K1) membranes expressing human CXCR2 receptors are purchased from Biosignal Packard Inc. All other chemicals are of analytical grade. White non-binding surface 96 well Optiplate™ microplates are obtained from Packard. Recombinant human IL-8 is synthesised, cloned and expressed in Escherichia coli as described previously (Lindley I, et al., Proc. Natl. Acad. Sci., 1988, 85(23):9199). The assay is performed in duplicate in 96 well Optiplate™ microplate in a final volume of 250 μl per well. Compounds are diluted in DMSO (0.5% final concentration) and incubated in 20 mM HEPES buffer pH 7.4 containing 10 mM MgCl₂, 100 mM NaCl, 1 mM EDTA plus 100 nM IL-8, 50 μM GDP and 500 pM [³⁵S]GTPγS per well. SPA beads (1 mg/well final concentration) were pre-mixed with the membranes (10 μg/well final concentration) in assay buffer: 20 mM HEPES buffer pH 7.4 containing 10 mM MgCl₂, 100 mM NaCl, 1 mM EDTA. The bead membrane mixture is added to each well, plates are sealed and incubated at room temperature for 60 minutes. The plate is centrifuged and read on Packard TopCount™ scintillation counter, program [³⁵S dpm] for 1 min/well. Data are expressed as the % response to 100 nM IL-8 minus basal.

### Chemotaxis Assay

The in vitro inhibitory properties of these compounds are determined in the neutrophil chemotaxis assay. Assays are performed in a 96-well plate format according to previously published method (Frevert C W, et al., J Immunolog. Methods, 1998, 213, 41). 96-well chemotaxis chambers 5 μm are obtained from Neuro Probe, all cell buffers are obtained from Invitrogen Paisley, UK, dextran -T500 and Ficoll-Paque Plus™ density gradient centrifugation media are purchased from Pharmacia Biotech Buckinghamshire, UK. Calcein-AM dye is obtained from Molecular Probes. Neutrophils are isolated as previously described (Haslett, C., et al. Am J Path., 1985, 119:101). Citrated whole blood is mixed with 4% (w/v) dextran-T500 and allowed to stand on ice for 30 minutes to remove erythrocytes. Granulocytes (PMN) are separated from peripheral blood mononuclear cells by layering 15 ml of cell suspension onto 15 ml Ficoll-Paque PLUS density gradient and centrifuged at 250×g for 25 minutes. Following centrifugation any erythrocytes contamination of PMN pellet is removed by hypotonic shock lysis using 10 ml ice-cold endotoxin-free sterile water for 50 seconds and neutralised with 10 ml of cold 2× phosphate buffered saline. Isolated neutrophils (1×10⁷) are labelled with the fluorochrome calcein-AM (5 μg) in a total volume of 1 ml and incubated for 30 minutes at 37° C. The labelled cells are washed with RPMI without phenol red+0.1% bovine serum albumin, prior to use the cells are counted and adjusted to a final concentration of 5×10⁶ cells/ml. The labelled neutrophils are then mixed with test compounds (0.001-1000 nM) diluted in DMSO (0.1% final concentration) and incubated for 10 minutes at room temperature. The chemoattractants (29 μl) are placed in the bottom chamber of a 96-well chemotaxis chamber at a concentration between (0.1-5 nM). The polycarbonate filter (5 μm) is overlaid on the plate, and the cells (25 μl) are loaded on the top filter. The cells are allowed to migrate for 90 minutes at 37° C. in a humidified incubator with 5% CO₂ At the end of the incubation period, migrated cells are quantified using a multi-well fluorescent plate reader (Fluoroskan II™, Labsystems) at 485 nm excitation and 538 nm emission. Each compound is tested in quadruplet using 4 different donors. Positive control cells, i.e. cells that have not been treated with compound, are added to the bottom well. These represent the maximum chemotactic response of the cells. Negative control cells, i.e. those that have not been stimulated by a chemoattractant, are added to the bottom chamber. The difference between the positive control and negative control represents the chemotactic activity of the cells.

The compounds of the Examples herein below generally have IC₅₀ values below 10 μM in the [³⁵S]-GPTγS binding assay. For instance, the compounds of Examples 1, 4, 7, 10, 14, 17, 22 and 23 have IC₅₀ values of 2.97, 0.66, 0.22, 4.89, 3.83, 0.99, 1.74 and 1.77 μM, respectively.

Having regard to their inhibition of binding of CXCR2, compounds of the invention are useful in the treatment of conditions or diseases mediated by CXCR2, for example inflammatory or allergic conditions or diseases, particularly chronic obstructive pulmonary airways or lung disease (COPD, COAD or COLD), including chronic bronchitis or dyspnea associated therewith, emphysema, bronchiolitis obliterans syndrome and severe asthma. Compounds of the present invention are further useful in the treatment of various diseases, such as cancer, e.g. ovarian cancer, prostate cancer, melanoma including metastatic melanoma, lung cancer, e.g. non small cell lung cancer, renal cell carcinoma; tumour angiogenesis, ischaemia/reperfusion injury, delayed graft function, osteoarthritis, myeloid metaplasia with myelofibrosis, Adenomyosis, contact hypersensitivity (skin). and in wound healing. Treatment in accordance with the invention may be symptomatic or prophylactic.

Prophylactic efficacy in the treatment of chronic bronchitis or COPD will be evidenced by reduced frequency or severity, will provide symptomatic relief and reduce disease progression, improvement in lung function. It may further be evidenced by reduced requirement for other, symptomatic therapy, i.e. therapy for or intended to restrict or abort symptomatic attack when it occurs, for example anti-inflammatory (e.g. corticosteroid) or bronchodilatory.

Other inflammatory or obstructive airways diseases and conditions to which the invention is applicable include acute lung injury (ALI), acute/adult respiratory distress syndrome (ARDS), idiopathic pulmonary fibrosis, fibroid lung, airway hyperresponsiveness, dyspnea, pulmonary fibrosis, allergic airway inflammation, small airway disease, lung carcinoma, acute chest syndrome in patients with sickle cell disease and pulmonary hypertension, as well as exacerbation of airways hyperreactivity consequent to other drug therapy, in particular other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds of the invention are also useful for treating respiratory viral infections, which exacerbate underlying chronic conditions such as asthma, chronic bronchitis, COPD, otitis media, and sinusitis. The respiratory viral infection treated may be associated with secondary bacterial infection, such as otitis media, sinusitis or pneumonia.

Compounds of the invention are also useful in the treatment of inflammatory conditions of the skin, for example psoriasis, atopic dermatitis, lupus erythematosus, and other inflammatory or allergic conditions of the skin.

Compounds of the invention may also be used for the treatment of other diseases or conditions, in particular diseases or conditions having an inflammatory component, for example, diseases affecting the nose including allergic rhinitis, e.g. atrophic, chronic, or seasonal rhinitis, inflammatory conditions of the gastrointestinal tract, for example inflammatory bowel disease such as ulcerative colitis and Crohn's disease, diseases of the bone and joints including rheumatoid arthritis, psoriatic arthritis, and other diseases such as atherosclerosis, multiple sclerosis, and acute and chronic allograft rejection, e.g. following transplantation of heart, kidney, liver, lung or bone marrow.

Compounds of the invention are also useful in the treatment of endotoxic shock, glomerulonephritis, cerebral and cardiac ischemia, Alzheimer's disease, cystic fibrosis, virus infections and the exacerbations associated with them, acquired immune deficiency syndrome (AIDS), multiple sclerosis (MS), Helicobacter pylori associated gastritis, and cancers, particularly the growth of ovarian cancer.

Compounds of the invention are also useful for treating symptoms caused by viral infection in a human which is caused by the human rhinovirus, other enterovirus, coronavirus, herpes viruses, influenza virus, parainfluenza virus, respiratory syncytial virus or an adenovirus.

The effectiveness of a compound of the invention in inhibiting inflammatory conditions, for example in inflammatory airways diseases, may be demonstrated in an animal model, e.g. mouse, rat or rabbit model, of airway inflammation or other inflammatory conditions, for example as described by Wada et al, J. Exp. Med. (1994) 180:1135-40; Sekido et al, Nature (1993) 365:654-57; Modelska et al., Am. J. Respir. Crit. Care. Med. (1999) 160:1450-56; and Laffon et al (1999) Am. J. Respir. Crit. Care Med. 160:1443-49.

The compounds of the invention are also useful as co-therapeutic compounds for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, fluticasone furoate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A_{2A} agonists such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, and WO 03/086408; and A_{2B} antagonists such as those described in WO 02/42298.

Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in EP 424021, U.S. Pat. No. 3,714,357, U.S. Pat. No. 5,171,744, WO 01/04118, WO 02/00652, WO 02/51841, WO 02/53564, WO 03/00840, WO 03/33495, WO 03/53966, WO 03/87094, WO 04/018422 and WO 04/05285; and beta-2 adrenoceptor agonists such as albuterol (salbutamol), metaproterenol, terbutaline, salmeterol fenoterol, procaterol, and especially, formoterol, carmoterol, milveterol and pharmaceutically acceptable salts thereof, and compounds (in free or salt or solvate form) of formula I of WO 00/75114, which document is incorporated herein by reference, preferably compounds of the Examples thereof, especially a compound of formula

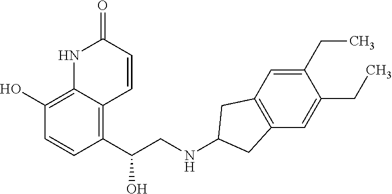

and pharmaceutically acceptable salts thereof, as well as compounds (in free or salt or solvate form) of formula I of WO 04/16601, and also compounds of EP 1440966, JP 05025045, WO 93/18007, WO 99/64035, US 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618 WO 04/46083 and WO 04/80964.

Suitable antihistamine drug substances include cetirizine hydrochloride, acetaminophen, clemastine fumarate, promethazine, loratidine, desloratidine, diphenhydramine and fexofenadine hydrochloride.

Combinations of compounds of the invention and anticholinergic or antimuscarinic compounds, steroids, beta-2 agonists, PDE4 inhibitors, dopamine receptor agonists, LTD4 antagonists or LTB4 antagonists may also be used. Other useful combinations of compounds of the invention with anti-inflammatory drugs are those with other antagonists of chemokine receptors, e.g. CCR-1, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzocyclohepten-8-yl]carbonyl]amino]phenyl]-methyl]-tetrahydro-N,N-dimethyl-2H-pyran-4-aminium chloride (TAK-770), CCR-5 antagonists described in U.S. Pat. No. 6,166,037 (particularly claims 18 and 19), WO 0066558 (particularly claim 8), and WO 0066559 (particularly claim 9).

In accordance with the foregoing, the invention also provides a method for the treatment of a condition or disease mediated by CXCR2, for example an inflammatory or allergic condition, particularly an inflammatory or obstructive airways disease, which comprises administering to a subject, particularly a human subject, in need thereof an effective amount of a compound of formula I in a free or pharmaceutically acceptable salt form as hereinbefore described. In another aspect the invention provides the use of a compound of formula I, in free or pharmaceutically acceptable salt form, as hereinbefore described for the manufacture of a medicament for the treatment of a condition or disease mediated by CXCR2, for example an inflammatory or allergic condition or disease, particularly an inflammatory or obstructive airways disease.

The compounds of the invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, for example in the treatment of atopic dermatitis; or rectally, for example in the treatment of inflammatory bowel disease.

In a further aspect, the invention also provides a pharmaceutical composition comprising as active ingredient a compound of formula I in free or pharmaceutically acceptable salt form, optionally together with a pharmaceutically acceptable diluent or carrier therefor. The composition may contain a co-therapeutic compound such as an anti-inflammatory bronchodilatory or antihistamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula I having a particle diameter up to 10 microns, optionally together with a diluent or carrier, such as lactose, of the desired particle size distribution and a compound that helps to protect against product performance deterioration due to moisture, e.g. magnesium stearate. When the composition comprises a nebulised formulation, it preferably contains, for example, the compound of formula I either dissolved, or suspended, in a vehicle containing water, a co-solvent such as ethanol or propylene glycol and a stabiliser, which may be a surfactant.

The invention includes (A) a compound of the invention in inhalable form, e.g. in an aerosol or other atomisable composition or in inhalable particulate, e.g. micronised form, (B) an inhalable medicament comprising a compound of the invention in inhalable form; (C) a pharmaceutical product comprising such a compound of the invention in inhalable form in association with an inhalation device; and (D) an inhalation device containing a compound of the invention in inhalable form.

Dosages of compounds of the invention employed in practising the present invention will of course vary depending, for example, on the particular condition to be treated, the effect desired and the mode of administration. In general, suitable daily dosages for administration by inhalation are of the order of 0.01 to 1 mg/kg per day while for oral administration suitable daily doses are of the order of 0.005 to 100 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily.

In the following examples all temperatures are in degree (°) Celsius.

The following ABBREVIATIONS are used:

AcOH acetic acid
aq. aqueous
DCC N,N′-dicyclohexylcarbodiimide
DMAP 4-dimethylaminopyridine

### DMA N,N-dimethylacetamide

### DMF N-N-dimethylformamide

DCM dichloromethane
EtOAc ethylacetate
HATU O-(7-Azabenzotriazol-1-yl)-N,N,N′,N′-tetramethyluronium hexafluorophosphate
mCPBA 3-chloroperoxybenzoic acid
sat. saturated
RT room temperature

¹H-NMR: Run on either Bruker Ultrashield™ 400 (400 MHz) spectrometer or are run on open Bruker AVANCE 400 NMR spectrometers using ICON-NMR. Spectra are measured at 298K and are referenced using the solvent peak, chemical shifts (δ-values) are reported in ppm, spectra splitting pattern are designated as singlet (s), doublet (d), triplet (t), quadruplet (q), multiplet or more overlapping signals (m), broad signal (br), solvent is given in parentheses.

### EXAMPLES

### Example 1

### 5-[2-(2-Fluoro-phenyl)-ethyl]-2-methyl-pyrazolo[1,5-a]pyrimidin-7-ol

0.16 g of 5-(2-Fluoro-phenyl)-3-oxo-pentanoicacid ethyl ester (Intermediate A1) and 0.065 g of 3-amino-5-methylpyrazole are suspended in 1 ml of AcOH under an atmosphere of argon and heated to 110°. The reaction mixture obtained is stirred at this temperature for 1.5 hours, allowed to cool to RT and 1 ml of H₂O is added. The precipitate obtained is collected by filtration washed with 1 ml of 1:1 AcOH/H₂O and dried to afford the title compound. ¹H NMR (400 MHz, DMSO-d6) 12.13 (1H, s) 7.32 (1H, m), 7.26 (1H, m), 7.12-7.17 (2H, m), 5.95 (1H, s), 5.48 (1H, s), 3.01 (2H, t) 2.83 (2H, t), 2.27 (3H, s).

Example 2 to 9 are prepared in an analagous way to Example 1 using the appropriately substituted phenyl-3-oxo-pentanoicacid ethyl esters (A Intermediates) and pyrazole/triazole amines (B Intermediates).

| Ex. | R₁ | R₂ | X | Y | MH⁺ |
|---|---|---|---|---|---|
| 1 | CH₃ | 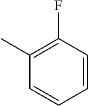 | C | C | 271.8 |
| 2 | NH₂ | 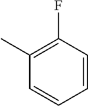 | N | C | 274 |
| 3 | 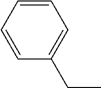 | 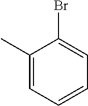 | C | C | 408.6 |
| 4 | 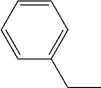 | 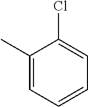 | C | C | 364.6 |
| 5 | 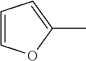 | 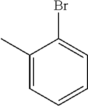 | C | C | 384.6 |
| 6 | 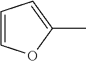 | 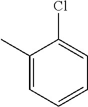 | C | C | 340.6 |
| 7 | 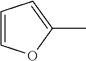 | 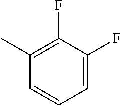 | C | C | 342.6 |
| 8 | CH₃ | 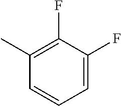 | C | C | 289.8 |
| 9 | 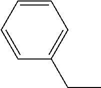 | 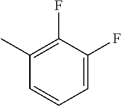 | C | C | 366.7 |

### Example 10

### 5-(2-Chloro-phenoxymethyl)-2-methyl-pyrazolo[1,5-a]pyrimidin-7-ol

A 60% dispersion of 0.061 g of NaH in mineral oil is suspended in 1 ml of dry DMF at 0-10° under an atmosphere of argon. 157 μl of 2-chlorophenol are added drop wise over a period of a few minutes, the ice-bath is removed and the reaction mixture obtained is stirred at RT for 45 minutes. The reaction mixture obtained is re-cooled to 0-10° and 0.1 g of 5-Chloromethyl-2-methyl-pyrazolo[1,5-a]pyrimidin-7-ol (Intermediate C1) are added. After warming to RT and stirring for 10 minutes the reaction mixture obtained is heated to 80° for 3 hours. On cooling the reaction mixture obtained is added to 30 ml of H₂O, a precipitate obtained is collected by filtration, washed with H₂O and dried under vacuum. Trituration of the solid obtained with iso-hexanes affords the title compound. ¹H NMR (400 MHz, DMSO-d6) 7.48 (1H, dd), 7.32 (1H, m), 7.20 (1H, dd), 7.02 (1H, m), 5.95 (1H, s), 5.74 (1H, s), 5.11 (2H, s), 2.28 (3H, s).

Examples 11 to 13 are obtained analogously to Example 10 by replacing 2-chlorophenol with the appropriate phenols.

| Ex. | R₁ | R₂ | X | Y | MH⁺ |
|---|---|---|---|---|---|
| 10 | CH₃ | 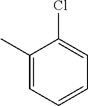 | C | O | 290.1 |
| 11 | CH₃ | 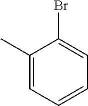 | C | O | 333.8 |
| 12 | CH₃ | 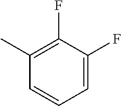 | C | O | 291.9 |
| 13 | CH₃ | 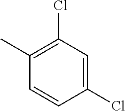 | C | O | 323.9 |

### Example 14

### 5-(2,6-Difluoro-phenoxymethyl)-2-furan-2-yl-pyrazolo[1,5-a]pyrimidin-7-ol

20 mg of 5-Chloromethyl-2-furan-2-yl-pyrazolo[1,5-a]pyrimidin-7-ol (Intermediate C2) and 10.4 g of 2,6-difluorophenol are dissolved in dry 300 μl of DMA. 0.055 g of finely ground anhydrous K₂CO₃ are added and the reaction mixture obtained is stirred in a heat block at 90° for 1.5 hours. 1.5 ml of glacial acetic acid are added and the suspension obtained is set aside for 15 minutes. Solvent is evaporated, 0.5 ml of MeOH and 5 ml of H₂O are added, a precipitate obtained is collected by filtration and washed with H₂O. The precipitate obtained is suspended in diethyl ether with stirring to give a solid. The solid obtained is filtered, washed with diethyl ether and dried under vacuum to afford the title compound. ¹H (400 MHz, DMSO-d₆): δ 5.16 (2H, s), 5.82 (1H, s), 6.43 (1H, s), 6.65 (1H, dd), 7.16-7.23 (3H, m), 7.83 (1H, d), 12.80 (1H, br s).

Examples 15 to 24 are prepared analogously to Example 14 by replacing 2,6-difluorophenol with the appropriate phenols.

| Ex. | R₁ | R₂ | X | Y | MH⁺ |
|---|---|---|---|---|---|
| 14 | 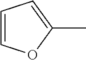 | 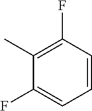 | C | O | 344.03 |
| 15 | 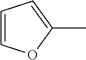 | 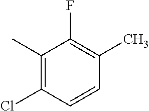 | C | O | 374.01 |
| 16 | 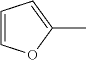 | 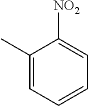 | C | O | 353.04 |
| 17 | 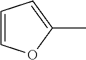 | 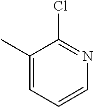 | C | O | 343.05 |
| 18 | 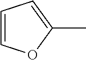 | 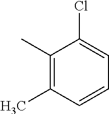 | C | O | 356.04 |
| 19 | 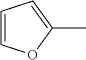 | 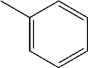 | C | O | 308.05 |
| 20 | 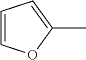 | 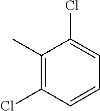 | C | O | 375.96 |
| 21 | 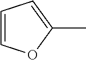 | 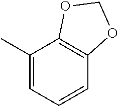 | C | O | 352.05 |
| 22 | 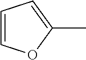 | 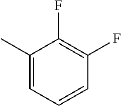 | C | O | 344.01 |
| 23 | 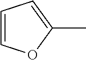 | 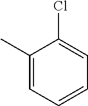 | C | O | 342.01 |
| 24 | 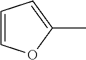 | 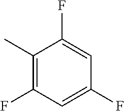 | C | O | 362.03 |

### Example 25

### 2-Methyl-5-(2,3,5,6-tetrafluoro-phenoxymethyl)-pyrazolo[1,5-a]pyrimidin-7-ol

This compound is prepared analogously to Example 1 by replacing 5-(2-Fluoro-phenyl)-3-oxo-pentanoicacid ethyl ester (Intermediate A1) with 3-Oxo-4-(2,3,5,6-tetrafluoro-phenoxy)-butyric acid ethyl ester (Intermediate A5) to afford the title compound.

### Example 26

### 2-Benzyl-5-(2,3,5,6-tetrafluoro-phenoxymethyl)[1,2,4]-triazolo[1,5-a]pyrimidin-7-ol

This compound is made analogously to Example 1 by replacing 5-(2-Fluoro-phenyl)-3-oxo-pentanoicacid ethyl ester (Intermediate A1) with 3-oxo-4-(2,3,5,6-tetrafluoro-phenoxy)-butyric acid ethyl ester (Intermediate A5) and 3-amino-5-methylpyrazole with 5-Benzyl-2H-[1,2,4]triazol-3-ylamine to afford the title compound.

| Ex. | R₁ | R₂ | X | Y | MH⁺ |
|---|---|---|---|---|---|
| 25 | CH₃ | 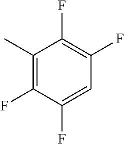 | C | O | 328.05 |
| 26 | 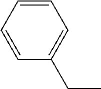 | 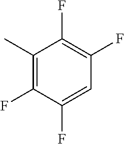 | N | O | 405.06 |

### Example 27

### 5-[2-(2,3-Difluoro-phenyl)-ethyl]-2-hydroxymethyl-pyrazolo[1,5-a]pyrimidin-7-ol

100 mg of 2-benzyloxymethyl-5-[2-(2,3-difluoro-phenyl)-ethyl]-pyrazolo[1,5-a]pyrimidin-7-ol (Intermediate F) is taken up in 10 ml methanol/AcOH (1:1). The solution is passed through a H-Cube hydrogenator at 1 bar pressure and 25° with a palladium hydroxide on carbon CatCart. The solvent is removed in vacuo and the residue is purified by flash chromatography on silica eluting with a 9:1 DCM/methanol mixture to afford the title compound. ¹H NMR (400 MHz, DMSO-d6) 12.20 (1H, br), 7.30 (1H, m), 7.15 (2H, m), 6.06 (1H, s), 5.52 (1H, s), 5.25 (1H, t), 4.50 (2H, d), 3.05 (2H, t), 2.85 (2H, t).

### Example 28

### 5-[2-(2,3-Difluoro-phenyl)-ethyl]-7-hydroxy-pyrazolo[1,5-a]pyrimidine-2-carboxylic acid

200 mg of 5-[2-(2,3-Difluoro-phenyl)-ethyl]-2-hydroxymethyl-pyrazolo[1,5-a]pyrimidin-7-ol (Example 27) is suspended in 5 ml water and 210 mg of potassium permanganate in 4 ml of 2M aqueous sodium hydroxide is added dropwise. After 1.5 hours the reaction mixture is filtered through Celite® (filter agent) and the filtrate extracted with DCM (2×40 ml). The aqueous extracts are acidified with 5M aqueous hydrochloric acid to precipitate a solid which is collected by suction filtration and dried to give the title compound. ¹H NMR (400 MHz, DMSO-d6) 13.20 (1H, br), 12.75 (1H, br), 7.25 (1H, m), 7.13 (2H, m), 6.48 (1H, s), 5.68 (1H, s), 3.05 (2H, t), 2.90 (2H, t).

### Example 29

### 5-[2-(2,3-Difluoro-phenyl)-ethyl]-7-hydroxy-pyrazolo[1,5-a]pyrimidine-2-carboxylic acid ethyl ester

290 mg of 5-[2-(2,3-Difluoro-phenyl)-ethyl]-7-hydroxy-pyrazolo[1,5-a]pyrimidine-2-carboxylic acid (Example 28) is suspended in 8 ml of ethanol and 0.5 ml of 2M aqueous hydrochloric acid added. The mixture is heated at reflux for 24 hours and then allowed to cool to RT and concentrated in vacuo. The residue is partitioned between EtOAc and H₂O and the aqueous extracted twice more with 40 ml of EtOAc. The combined EtOAc extracts washed with 50 ml brine then dried over magnesium sulfate, filtered and the solvent evaporated. The residue is purified by flash chromatography on silica eluting with a 1:1 iso-hexane/EtOAc to yield the title compound. ¹H NMR (400 MHz, DMSO-d6) 12.35 (1H, br), 7.25 (1H, m), 7.13 (2H, m), 6.50 (1H, s), 5.68 (1H, s), 4.31 (2H, q), 3.05 (2H, t), 2.90 (2H, t), 1.32 (3H, t).

### Example 30

### 5-[2-(2,3-Difluoro-phenyl)-ethyl]-7-hydroxy-pyrazolo[1,5-a]pyrimidine-2-carbaldehyde

To 1.0 g of 5-[2-(2,3-Difluoro-phenyl)-ethyl]-2-hydroxymethyl-pyrazolo[1,5-a]pyrimidin-7-ol (Example 27) in 6 ml of DMF and 12 ml of chloroform is added 2.28 g of manganese (IV) oxide in one portion. The mixture is heated at 60° for 7 hours and then is allowed to cool to RT. The reaction mixture is then filtered through Celite® (filter agent) and the filtrate evaporated in vacuo and the residue purified by flash chromatography on silica eluting with DCM to 12:1 DCM/methanol to afford the title compound. ¹H NMR (400 MHz, DMSO-d6) 12.75 (1H, br), 10.00 (1H, s), 7.25 (1H, m), 7.13 (2H, m), 6.45 (1H, s), 5.65 (1H, s), 3.05 (2H, t), 2.86 (2H, t).

### Example 31

### 5-[2-(2,3-Difluoro-phenyl)-ethyl]-7-hydroxy-pyrazolo[1,5-a]pyrimidine-2-carbaldehyde oxime

215 mg of 5-[2-(2,3-Difluoro-phenyl)-ethyl]-7-hydroxy-pyrazolo[1,5-a]pyrimidine-2-carbaldehyde (Example 30) is suspended in 8 ml of ethanol and 93 mg of sodium acetate added followed by 59 mg of hydroxylamine hydrochloride dissolved in 4 ml of H₂O. The resulting solution is heated at reflux for 3 hours and then allowed to cool to room temperature and concentrated in vacuo. The residue is purified by flash chromatography on silica eluting with 9:1 DCM/methanol to afford the title compound. ¹H NMR (400 MHz, DMSO-d₆) 12.30 (1H, br), 11.58 (1H, s), 8.10 (1H, s), 7.28 (1H, m), 7.12 (2H, m), 6.23 (1H, s), 5.57 (1H, s), 3.05 (2H, t), 2.85 (2H, t).

### Example 32

### 5-[2-(2,3-Difluoro-phenyl)-ethyl]-7-hydroxy-pyrazolo[1,5-a]pyrimidine-2-carboxylic acid (1H-tetrazol-5-yl) amide

100 mg of 5-[2-(2,3-Difluoro-phenyl)-ethyl]-7-hydroxy-pyrazolo[1,5-a]pyrimidine-2-carboxylic acid (Example 28) is suspended in 3 ml of dry DMF were added 55 mg of carbonyl diimidazole and 30 mg of 5-aminotetrazole. The mixture is allowed to stir for 72 hours and then concentrated to dryness in vacuo. The residue is triturated with methanol and the beige solid is collected by suction filtration to yield the title compound. ¹H NMR (400 MHz, DMSO-d6) 13.25 (2H, br), 7.90 (1H, s), 7.28 (1H, m), 7.12 (2H, m), 6.72 (1H, s), 5.72 (1H, s), 3.05 (2H, t), 2.90 (2H, t).

### Example 33

### N-{5-[2-(2,3-Difluoro-phenyl)-ethyl]-7-hydroxy-pyrazolo[1,5-a]pyrimidine-2-carbonyl}-benzenesulfonamide

50 mg of 5-[2-(2,3-Difluoro-phenyl)-ethyl]-7-hydroxy-pyrazolo[1,5-a]pyrimidine-2-carboxylic acid (Example 28) is dissolved in 2 ml of dry DMF. 140 mg of HATU, 77 mg DMAP and 50 mg benzene sulfonamide. The mixture is stirred for 18 hours and then the reaction mixture is added to 25 ml EtOAc. This was washed with 25 ml 1M aqueous HCl. The organic phase is removed and washed with 25 ml brine, dried over magnesium sulfate, filtered and evaporated. The residue was triturated with methanol and the resulting solid removed by vacuum filtration and dried to afford the title compound. ¹H NMR (400 MHz, DMSO-d6) 12.69 (1H, s), 8.08 (2H, m), 7.79 (1H, m), 7.70 (1H, t), 7.34 (1H, m), 7.19 (2H, m), 6.59 (1H, s), 5.79 (1H, s), 3.13 (2H, t), 2.95 (2H, t).

### Example 34

### 5-[2-(2,3-Difluoro-phenyl)-ethyl]-7-hydroxy-pyrazolo[1,5-a]pyrimidine-2-carbonitrile

To 160 mg of 5-[2-(2,3-Difluoro-phenyl)-ethyl]-7-hydroxy-pyrazolo[1,5-a]pyrimidine-2-carbaldehyde oxime (Example 31) in 5 ml of acetic anhydride is added 50 mg of sodium acetate. The mixture is heated at 100° for 18 hours. After allowing to cool to RT the reaction mixture is partitioned between H₂O and EtOAc. The water layer is removed and extracted twice more with EtOAc. The combined EtOAc extracts are washed with 50 ml brine then dried over magnesium sulfate, filtered and the solvent evaporated. The residue is purified by flash chromatography on silica eluting with a DCM to 15:1 DCM/methanol to yield the title compound. ¹H NMR (400 MHz, MeOD) 7.12 (3H, m), 6.65 (1H, s), 5.78 (1H, s), 3.15 (2H, t), 2.97 (2H, t)

### Example 35

### 5-[2-(2,3-Difluoro-phenyl)-ethyl]-2-(1H-tetrazol-5-yl)-pyrazolo[1,5-a]pyrimidin-7-ol

50 mg of 5-[2-(2,3-Difluoro-phenyl)-ethyl]-7-hydroxy-pyrazolo[1,5-a]pyrimidine-2-carbonitrile (Example 34) dissolved in 6 ml of dry DMF are added 22 mg of sodium azide and 18 mg of ammonium chloride. The mixture is heated at 120° for 48 hours. After allowing to cool the solvent was removed in vacuo. The residue was purified by flash chromatography on silica eluting with DCM to 4:1 DCM/methanol to yield the title compound. ¹H NMR (400 MHz, MeOD) 7.12 (3H, m), 6.80 (1H, s), 5.72 (1H, s), 3.15 (2H, t), 3.02 (2H, t).

| Ex. | R₁ | R₂ | X | Y | MH⁺ |
|---|---|---|---|---|---|
| 27 | 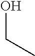 | 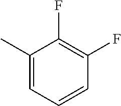 | C | C | 305.8 |
| 28 | 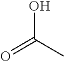 | 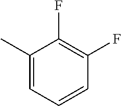 | C | C | 320.2 |
| 29 | 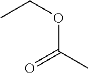 | 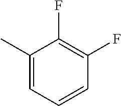 | C | C | 348.0 |
| 30 | 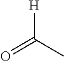 | 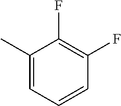 | C | C | 304.0 |
| 31 | 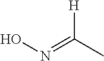 | 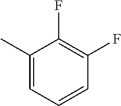 | C | C | 333.1 |
| 32 | 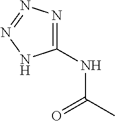 | 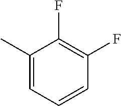 | C | C | 386.8 |
| 33 | 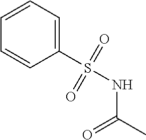 | 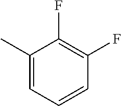 | C | C | 459.0 |
| 34 | 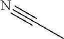 | 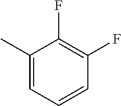 | C | C | 301.21 |
| 35 | 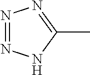 | 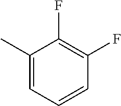 | C | C | 344.3 |

### Example 36

### N-{5-[2-(2,3-Difluoro-phenyl)-ethyl]-7-hydroxy-pyrazolo[1,5-a]pyrimidin-2-yl}acetamide

1.0 g of 1H-Pyrazole-3,5-diamine (Intermediate G) is dissolved in 15 ml of AcOH. 3.0 g of 5-(2,3-difluoro-phenyl)-3-oxo-pentanoic acid ethyl ester (Intermediate A4) added and the solution is heated at 100° for 18 hours. The reaction is then cooled to 0° and the solids collected by suction filtration and washed well with AcOH and diethyl ether and then dried under vacuum to yield the title compound. ¹H NMR (400 MHz, DMSO-d6) 12.10 (1H, br), 10.88 (1H, s), 7.28 (1H, m), 7.12 (2H, m), 6.41 (1H, s), 5.52 (1H, s), 3.05 (2H, t), 2.85 (2H, t), 2.02 (3H, s).

### Example 37

### 2-Amino-5-[2-(2,3-difluoro-phenyl)-ethyl]-pyrazolo[1,5-a]pyrimidin-7-ol

2.0 g of N-{5-[2-(2,3-Difluoro-phenyl)-ethyl]-7-hydroxy-pyrazolo[1,5-a]pyrimidin-2-yl}acetamide (Example 36) is suspended in 60 ml methanol and 10 ml H₂O. 3 ml of 5 M aqueous hydrochloric acid is added and the mixture heated at 80° for 18 hours. Solution is attained during this time. The reaction is then allowed to cool to RT and made to pH 8 with 2 M aqueous sodium hydroxide. The resulting precipitate is collected by suction filtration and dried under vacuum to give the title compound. ¹H NMR (400 MHz, DMSO-d6) 7.28 (1H, m), 7.12 (2H, m), 5.45 (1H, br), 5.20 (2H, br), 4.92 (2H, br), 3.05 (2H, t), 2.75 (2H, t).

### Example 38

### N-{5-[2-(2,3-Difluoro-phenyl)-ethyl]-7-hydroxy-pyrazolo[1,5-a]pyrimidin-2-yl}benzenesulfonamide

50 mg of 2-Amino-5-[2-(2,3-difluoro-phenyl)-ethyl]-pyrazolo[1,5-a]pyrimidin-7-ol (Example 37) is dissolved in 4 ml of dry pyridine. 4 mg of N,N-dimethylaminopyridine is added followed by 33 μl of benzenesulfonyl chloride. After stirring for 18 hours the pyridine is removed in vacuo and the residue taken up in 50 ml DCM. This is washed with 3×25 ml of 2 M aqueous hydrochloric acid, 20 ml H₂O, 20 ml brine. Then dried over MgSO₄, filtered and evaporated in vacuo. The residue is purified by flash chromatography on silica eluting with 15:1 DCM/methanol to give the title compound. ¹H NMR (400 MHz, DMSO-d6) 12.15 (1H, br), 11.35 (1H, br), 7.85 (2H, d), 7.58 (3H, m), 7.25 (1H, m), 7.10 (2H, m), 5.85 (1H, s), 5.50 (1H, s), 3.05 (2H, t), 2.75 (2H, t).

| Ex. | R₁ | R₂ | X | Y | MH⁺ |
|---|---|---|---|---|---|
| 36 | 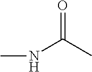 | 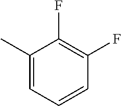 | C | C | 333.1 |
| 37 | —NH₂ | 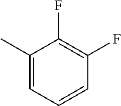 | C | C | 291.0 |
| 38 | 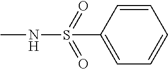 | 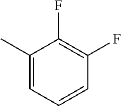 | C | C | 431.3 |

### Preparation of Intermediates

### Intermediate A1: 5-(2-Fluoro-phenyl)-3-oxo-pentanoicacid ethyl ester

### Step 1: 5-[3-(2-Fluoro-phenyl)-propionyl]-2,2-dimethyl-[1,3]dioxane-4,6-dione

2.0 g of 3-(2-Fluoro-phenyl)-propionic acid and 2.45 g of DCC are suspended in 30 ml of dry DCM under an atmosphere of argon. To the mixture obtained 1.71 g of meldrum's acid 1.45 g of DMAP are added, a dispersion is obtained and stirred overnight at RT. The reaction mixture obtained is filtered and washed with a small amount of DCM, the filtrate obtained is reduced in vacuo. The product obtained is dissolved in 50 ml of EtOA and re-filtered, the filtrate obtained is washed with 30 ml of 1M aq. HCl and brine. dried over MgSO₄ and concentrated in vacuo to afford the title compound.

### Step 2: 5-(2-Fluoro-phenyl)-3-oxo-pentanoicacid ethyl ester

3.25 g of 5-[3-(2-Fluoro-phenyl)-propionyl]-2,2-dimethyl-[1,3]dioxane-4,6-dione are dissolved in 30 ml of EtOH under an atmosphere of argon and heated to reflux. After about 3 hours the reaction mixture obtained is reduced in vacuo and dried to afford the title compound.

Intermediates A2-A5 are prepared analogously to Intermediate A1 by replacing 3-(2-Fluoro-phenyl)-propionic acid with the appropriate phenylpropionic acids.

These compounds namely are:
A2: 5-(2-Chloro-phenyl)-3-oxo-pentanoicacid ethyl ester
A3: 5-(2-Bromo-phenyl)-3-oxo-pentanoic acid ethyl ester
A4: 5-(2,3-Difluoro-phenyl)-3-oxo-pentanoic acid ethyl ester
A5: 3-Oxo-4-(2,3,5,6-tetrafluoro-phenoxy)-butyric acid ethyl ester

### Intermediate B1: 5-Benzyl-2H-pyrazol-3-ylamine

### Step 1: 2-Oxo-3-phenyl-propionitrile

1.56 ml of dry ACN are added to a stirred suspension of 60% NaH in mineral oil in 75 ml of 1,4-dioxane under an atmosphere of argon. The reaction mixture obtained is stirred at RT for 20 minutes and a solution of 3.98 ml of phenyl-acetic acid ethyl ester in 35 ml of 1,4-dioxane is added and the reaction mixture obtained is heated to reflux for 4 hours. On cooling, 75 ml of H₂O and DCM are added, the aqueous phase obtained is separated and washed with 50 ml of DCM. The aqueous phase obtained is acidified to pH5 with 2M HCl, solvent of the precipitate obtained is evaporated and the title compound is obtained.

### Step 2: 5-Benzyl-2H-pyrazol-3-ylamine

0.336 ml of Hydrazine hydrate are added to a stirred solution of 1.2 g of 2-Oxo-3-phenyl-propionitrile in 60 ml of EtOH. The reaction mixture obtained is heated to reflux for 4 hours, cooled to RT and solvent is evaporated. The title compound is obtained.

### Intermediate C1: 5-Chloromethyl-2-methyl-pyrazolo[1,5-a]pyrimidin-7-ol

0.343 g of 3-amino-5-methylpyrazole are dissolved in 4 ml of AcOH under an atmosphere of argon at RT. 0.408 ml of 4-Chloro-3-oxo-butyric acid methyl ester are added and the reaction mixture obtained is heated to 110° for 10 minutes and a precipitate is obtained. On cooling to RT the reaction mixture obtained is diluted with 4 ml of MeCN, the precipitate obtained is collected by filtration, the filtrate obtained is washed with MeCN, solvent is evaporated and the title compound is obtained [MH+198.1].

### Intermediate C2: 5-Chloromethyl-2-furan-2-yl-pyrazolo[1,5-a]pyrimidin-7-ol

This compound is obtained analogous to Intermediate Cl by replacing 3-amino-5-methylpyrazole with 3-amino-5-(2-furyl)pyrazole to afford the title compound.

### Intermediate D1: Benzo[1,3]dioxol-4-ol

### Step 1: Benzo[1,3]dioxole-4-carbaldehyde

25 g of 2,3-dihydroxy-benzaldehyde, 36 ml of dibromomethane, 72 g of K₂CO₃, 0.752 g of copper(II)oxide and 1000 ml of DMF are mixed together and heated to reflux for 4 hours. The reaction mixture obtained is cooled to RT, a filtrate is obtained and solvent is evaporated. The residue obtained is dissolved in toluene and the organic layer obtained is washed with sat. aq. NaHCO₃ and brine. The organic layer obtained is dried over MgSO₄ and from the filtrate obtained solvent is evaporated. The title compound is obtained.

### Step 2: Formic acid benzo[1,3]dioxol-4-yl ester

To a solution of 23.5 g of Benzo[1,3]dioxole-4-carbaldehyde in 250 ml of DCM, 40.3 g of mCPBA are added in small portions over a period of 15 minutes and the reaction mixture obtained is stirred at 40° C. for 18 hours. On cooling, the reaction mixture obtained is concentrated in vacuo and the residue obtained is dissolved in EtOAc and washed 4× with sat. aq. NaHCO₃ and brine. The organic layer obtained is dried over MgSO₄, filtered and solvent is evaporated to afford the title compound.

### Step 3: Benzo[1,3]dioxol-4-ol

19 g of Formic acid benzo[1,3]dioxol-4-yl ester are dissolved in 20 ml of MeOH, 90 ml of KOH (10% in water) are added. The reaction mixture obtained is stirred for 1 hour at RT, the solution obtained is acidified with conc. HCl and extracted with diethyl ether. The organic layer obtained is washed with brine, dried over MgSO₄, filtered and solvent is evaporated. Purification by recrystallisation from DCM/hexane affords the title compound.

### Intermediate E: 5-Benzyloxymethyl-2H-pyrazol-3-ylamine

### Step 1: Benzyloxy-acetic acid ethyl ether

11.5 g of ethyl glycolate is dissolved in 120 ml of dry THF under an atmosphere of nitrogen and cooled to 0°. 4.9 g of sodium hydride is added portionwise over 40 minutes and the reaction mixture stirred at 0°° for 15 minutes and then 4.3 g of tetrabutylammonium iodide is added followed by 13.3 ml of benzyl bromide. The mixture allowed to warm to RT and stirred for 3 hours. The reaction then quenched with 20 ml of a saturated aqueous ammonium chloride solution and the THF then removed in vacuo. The residue is partitioned between EtOAc (100 ml) and H₂O (150 ml). The aqueous then extracted twice with EtOAc and the combined EtOAc extracts washed with 50 ml brine then dried over magnesium sulfate, filtered and the solvent evaporated. The residue is purified by flash chromatography on silica eluting with a 19:1 iso-hexane/diethyl ether to 4:1 iso-hexane/diethyl ether mixture to yield the title compound.

### Step 2: 4-Benzyloxy-3-oxo-butyronitrile

4.2 ml of dry acetonitonitrile in 170 ml of dry THF under an atmosphere of nitrogen is cooled to −78°. 52 ml of a 1.6 M butyllithium solution in hexanes then added dropwise over 45 minutes. After stirring at −78° for a further 15 minutes 14.0 g of benzyloxy-acetic acid ethyl ether is added dropwise. The reaction then allowed to warm to RT and stirred for 2 hours. The reaction mixture is then poured into 200 ml of H₂O. The THF and hexanes then removed in vacuo and the residue made to pH 5 with a 1M aqueous hydrochloric acid and then extracted with EtOAc (3×100 ml). The combined EtOAc extracts washed with 50 ml brine then dried over magnesium sulfate, filtered and the solvent evaporated to obtain the title compound.

### Step 3: 5-Benzyloxymethyl-2H-pyrazol-3-ylamine

13.3 g of 4-Benzyloxy-3-oxo-butyronitrile is taken up in 150 ml ethanol and 18 ml of hydrazine monohydrate is added. The mixture is heated at reflux for 10 hours, cooled to RT and the solvent is evaporated. The residue is purified by flash chromatography on silica eluting with 5% methanol in chloroform to afford the title compound.

### Intermediate F: 2-Benzyloxymethyl-5-[2-(2,3-difluoro-phenyl)-ethyl]-pyrazolo[1,5-a]pyrimidin-7-ol

3.45 g of 5-benzyloxymethyl-2H-pyrazol-3-ylamime (Intermediate E) is taken up in 50 ml AcOH and 5.0 g of 5-(2,3-difluoro-phenyl)-3-oxo-pentanoic acid ethyl ester (Intermediate A4) added. The reaction mixture is heated at reflux for 10 hours and then allowed to cool to RT and the solvent is evaporated in vacuo. The residue is triturated with diethyl ether to afford the title compound.

### Intermediate G: 1H-Pyrazole-3,5-diamine

24.5 g of diethyl malonimidate dichloride in 200 ml of diethyl ether is cooled to 0° and 200 ml of an aqueous saturated K₂CO₃ is added and the mixture stirred vigorously for 5 minutes. The organic layer was removed and the aqueous extracted twice more with diethyl ether. The combined diethyl ether extracts are dried over magnesium sulfate, filtered and evaporated in vacuo. The resulting oil is added portionwise to 5 ml of hydrazine monohydrate in 50 ml ethanol and heated at reflux for 15 minutes. The reaction is then cooled to 5° and allowed to stand for 18 hours. The resulting solid precipitated is collected by suction filtration and dried under vacuum to afford the title compound.

1. A compound of formula (I) 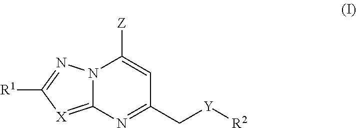
wherein
R¹ is a group of the formula: -A-(C₀-C₈ alkylene)-B;
A is a bond, —C(O)N(R^{a})—, —C(O)NHS(O)—, —C(O)NHS(O₂)—, —C(O)—, —C(O)O—, —C(O)-(5 or 6-membered N-bonded heterocyclic bridging group)-, —N(R^{a})C(O)—, —(CH₂)_{z}—N(R^{a})—, —(CH₂)_{z}—N(R^{a})S(O)—, —(CH₂)_{z}—N(R^{a})S(O₂)—, —C(═N—OR^{a})— or —NHC(═NH)N(R^{a})—;
B is H, OH, CN, NO₂, halogen, C₁-C₈ alkylthio, C₂-C₆ alkenyl, C₂-C₆ alkynyl, C₃-C₈ cycloalkyl, C₅-C₈ cycloalkenyl, C₆-C₁₄ aryl, a 5-10 membered heterocyclic group containing one or more heteroatoms selected from N, O and S, C₁-C₆ alkoxy, O—C₃-C₈ cycloalkyl, O—C₁-C₃ alkylene-C₃-C₈ cycloalkyl, O—C₆-C₁₄ aryl, O-benzyl, O-(5-10 membered heterocyclic group containing one or more heteroatoms selected from N, O and S), C(O)R^{d}, C(O)OR^{b}, OC(O)R^{b}, C(O)NR^{b}R^{c}, N(R^{b})C(O)R^{d}, NR^{b}R^{c}, S(O)C₁-C₆ alkyl or S(O₂)C₁-C₆ alkyl, wherein the alkyl, alkenyl and alkynyl groups are each optionally substituted by OH, halo or C₁-C₃ alkoxy, wherein the cycloalkyl and cycloalkenyl groups are each optionally fused to a benzene ring and the ring as a whole is optionally substituted by OH, halo, NH₂ or C₁-C₃ alkoxy, and wherein the aryl and heterocyclic groups are each optionally substituted by one or substituents each independently selected from OH, halo, NH₂, CN, NO₂, oxo, C₁-C₆ alkyl, C₁-C₆ hydroxyalkyl, C₁-C₆ haloalkyl, C₃-C₈ cycloalkyl, C₁-C₆ alkoxy, C₁-C₆ haloalkoxy, phenyl, a 5-10 membered heterocyclic group containing one or more heteroatoms selected from N, O and S and CO₂R^{b};
the (C₀-C₈ alkylene group) may be branched and is optionally substituted by OH or C₁-C₃ alkoxy;
z is 0, 1, 2 or 3;
R^{a} and R^{b} are each independently selected from H, C₁-C₆ alkyl, C₃-C₈ cycloalkyl and C₅-C₈ cycloalkenyl;
R^{c} and R^{d} are each independently selected from H, C₁-C₆ alkyl, C₃-C₈ cycloalkyl, C₅-C₈ cycloalkenyl, C₆-C₁₄ aryl, a 5-10 membered heterocyclic group containing one or more heteroatoms selected from N, O and S, S(O)C₁-C₆ alkyl and S(O₂)C₁-C₆ alkyl,
provided that R¹ is not hydrogen;
R² is C₆-C₁₄ aryl, —C₁-C₆ alkylene-C₆-C₁₄ aryl or a 5-10 membered heterocyclic group containing one or more heteroatoms selected from N, O and S, wherein the aryl and heterocyclic groups are each optionally fused to a 5 or 6-membered non-aromatic carbocyclic group or a 5 or 6-membered non-aromatic heterocyclic group containing one or more heteroatoms selected from N, O and S and wherein the ring systems are optionally substituted by OH, halo, NH₂, CN, NO₂, oxo, C₁-C₆ alkyl, C₁-C₆ hydroxyalkyl, C₁-C₆ haloalkyl, C₃-C₈ cycloalkyl, C₁-C₆ alkoxy, C₁-C₆ haloalkoxy, phenyl, a 5-10 membered heterocyclic group containing one or more heteroatoms selected from N, O and S and CO₂R^{b};
X is C or N;
Y is O or CH₂;
Z is OR³ or NR³R⁴;
R³ is H, C₁-C₆ alkyl, C₃-C₈ cycloalkyl or C₆-C₈ cycloalkenyl;
R⁴ is H, C₁-C₆ alkyl, C₃-C₈ cycloalkyl and C₅-C₈ cycloalkenyl, wherein the alkyl and cycloalkyl groups are each optionally substituted by one or more groups selected from OH and C₁-C₃ alkoxy;
or a pharmaceutically acceptable salt or solvate thereof.

2. A compound of claim 1, wherein
R² is phenyl, optionally fused with a 5 membered non-aromatic heterocyclic group containing 2 oxygen heteroatoms, wherein the ring system is optionally substituted by one or more groups selected from C₁-C₃ alkyl and halogen; or
pyridinyl optionally substituted by one or more halogen atoms.

3. A compound of any one of claim 1, wherein
X is C.

4. A compound of any one of claims 1 in combination with at least one second drug substance.

5-7. (canceled)

8. A method for the prevention or treatment of a CXCR2 receptor mediated condition or disease comprising administering an effective amount of at least one compound according to claim 1 to a subject in need of such treatment.

9. A method of claim 8 wherein the condition or disease is an inflammatory or allergic condition, particularly an inflammatory or obstructive airway disease.

10. Use of a compound of formula I as defined in claim 1 in the manufacture of a medicament for the treatment of an inflammatory or allergic condition or disease, particularly an inflammatory or obstructive airway disease.

11. A process for the preparation of a compound of formula (I) according to claim 1, the process comprising:
(a) reacting a compound of formula (A) 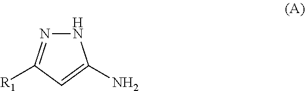
with a compound of formula (B) 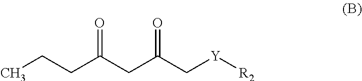
wherein R¹, R² and Y are as defined in claim 1; or
(b) reacting a compound of formula 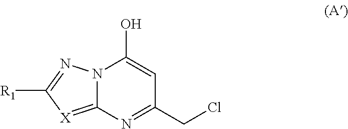
with a compound of formula R₂OH,
wherein R¹, R² and X are as defined in claim 1.
